(19) United States
(12) Patent Application Publication
(10) Pub. No.: US 2007/0150020 A1
(43) Pub. Date: Jun. 28, 2007

(54) Externally oriented battery feedthrough with integral connector

(76) Inventors: Karl E. Hokanson, Coon Rapids, MN (US); Paul B. Aamodt, Howard Lake, MN (US); Andrew J. Ries, Lino Lakes, MN (US)

Correspondence Address: MEDTRONIC, INC., 710 MEDTRONIC PARK, MINNEAPOLIS, MN, 55432-9924, US

(21) Appl. No.: 11/320,357

(22) Filed: Dec. 28, 2005

(51) Int. Cl. A61N 1/00

(52) U.S. Cl. 607/30

(57) ABSTRACT

A battery for use with implantable medical devices, and a method of making the battery. The battery includes a battery housing, a connector block connected to the battery housing, a feedthrough assembly having a ferrule, where at least a portion of the ferrule extends outside the battery housing, and within the connector block.

### BACKGROUND OF THE INVENTION

The present invention relates to volumetrically efficient batteries for use with implantable medical devices. Implantable medical devices (IMDs), such as implantable pacemakers and implantable cardioverter-defibrillators (ICDs), are electronic medical devices that monitor the electrical activity of the heart and provide therapy in the form of electrical stimulation to one or more of the heart chambers. Pacemakers and ICDs are designed with shapes that are conforming to the patient's body. Minimizing the volume occupied by the devices is an ongoing effort to enhance patient comfort. Accordingly, the trend in the field of implantable medical devices is to provide devices that are thinner, smaller, and lighter.

In order to perform pacing and/or cardioversion-defibrillation functions, IMDs require an energy source. The battery of an IMD typically requires allocation of a substantial volume within the implantable medical device. Reducing the volume of the battery generally results in a corresponding reduction in battery capacity. A reduction in battery capacity, however, can result in a shorter operating life of an IMD. Thus, there is an ongoing need to provide batteries for IMDs having reduced volumes without corresponding reductions in battery capacity.

### BRIEF SUMMARY OF THE INVENTION

The disclosure relates to a battery for use with implantable medical devices, and a method of making the battery. The battery includes a battery housing, a connector block, and a feedthrough assembly, where the feedthrough assembly includes a ferrule that is disposed at least partially outside of the battery housing, and within the connector block. This increases the volumetric efficiency of the battery without reducing capacity.

### BRIEF DESCRIPTION OF THE DRAWINGS

FIG. 1 is an exploded front perspective view of a battery assembly, which includes a battery disposed between insulative housings.

FIG. 2 is an exploded rear perspective view of the battery.

FIG. 3 is a top rear perspective view of the battery assembly, with a portion broken away to show a connection between cathode tabs of an electrode assembly and a feedthrough pin of a feedthrough assembly.

FIG. 4 is an expanded view of the battery, illustrating the interaction between the feedthrough assembly and a connector block.

FIGS. 5A and 5B are sectional views of section 5-5 taken in FIG. 4, depicting the feedthrough assembly and the connector block secured to a battery housing.

FIG. 6 is a block diagram illustrating a method of manufacturing the battery.

### DETAILED DESCRIPTION

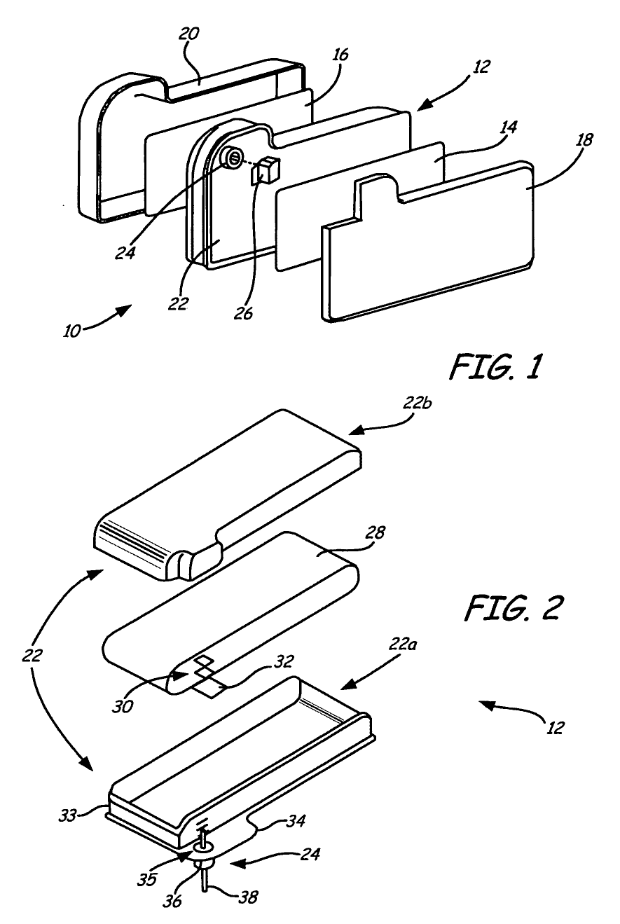

FIG. 1 is an exploded front perspective view of battery assembly 10, which includes battery 12, adhesive layers 14 and 16, and insulative housings 18 and 20. Battery 12 has increased volumetric efficiency while preserving battery capacity, and without increasing the overall volume of battery assembly 10. Battery 12 includes battery housing 22, feedthrough assembly 24, and connector block 26, where feedthrough assembly 24 is secured to battery housing 22 within an aperture in battery housing 22. Connector block 26 encases feedthrough assembly 24 and is also connected to battery housing 22. Feedthrough assembly 24 and connector block 26 are the components of battery 12 that connect to circuitry within an IMD (not shown).

As discussed below, a portion of feedthrough assembly 24 extends at least partially outside of battery housing 22, and within connector block 26. This increases the amount of free space within battery housing 22 without reducing the capacity of battery 12. As a result, battery 12 may incorporate additional active battery components to increase capacity. Alternatively, battery housing 22 may have a more compact design to reduce the overall volume of battery assembly 10. Placing feedthrough assembly 24 within connector block 26 also makes efficient use of the volume within connector block 26.

Adhesive layers 14 and 16 secure insulative housings 18 and 20 to battery 12. Insulative housings 18 and 20 encase battery 12 so that electrical power from battery 12 is routed through feedthrough assembly 24 and connector block 26. In alternative embodiments, battery assembly 10 may incorporate a variety of different insulation components, such as insulative adhesive layers, which are also beneficial for adhering battery 12 to circuitry and housings of IMDs. Battery assembly 10 may also have differing designs from that shown in FIG. 1 (e.g., deep prismatic designs). Accordingly, battery assembly 10 may be employed in a variety of electronic and mechanical devices for treating patient medical conditions, such as pacemakers, ICDs, neurostimulators, and therapeutic-substance delivery pumps.

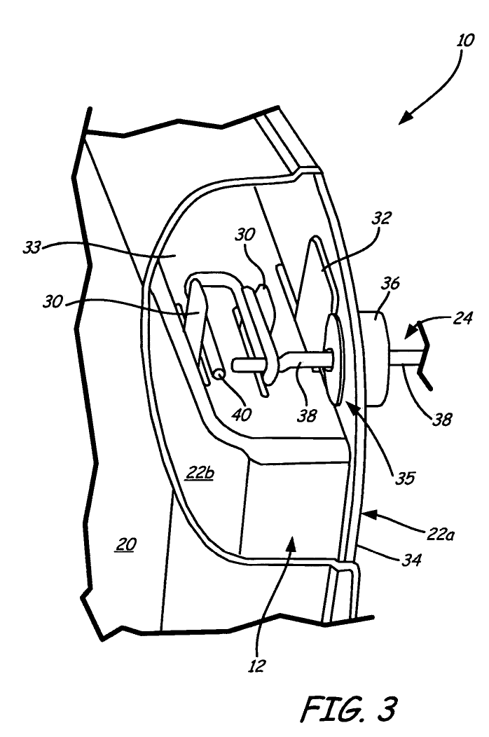

FIG. 2 is an exploded rear perspective view of battery 12, which further includes electrochemical cell 28 disposed between front housing 22a and rear housing 22b of battery housing 22. Electrochemical cell 28 is a coiled, wound, folded, or stacked cell structure of an electrochemical cell, which stores electrical energy for operating implantable medical devices. As shown, electrochemical cell 28 includes cathode tabs 30, and anode tab 32, which are electrodes respectively connected to a cathode portion and an anode portion of electrochemical cell 28.

Front housing 22a includes insulative cup 33 and conductive cover 34, where insulative cup 33 is secured to conductive cover 34. Rear housing 22b includes an insulative caseliner (not shown) disposed within a conductive outer casing (electrolyte fill port not shown). Suitable materials for insulative cup 33 and the insulative caseliner of rear housing 22b include electrically-insulative plastics, such as ethylene-tetrafluoroethylenes. Suitable materials for conductive cover 34 and the conductive outer casing of rear housing 22b include conductive materials, such as titanium.

As further shown in FIG. 2, conductive cover 34 includes aperture 35, which is an annular orifice through which feedthrough assembly 24 extends. Feedthrough assembly 24 includes ferrule 36 and feedthrough pin 38. Ferrule 36 is an annular, electrically-conductive collar that extends within aperture 35. In alternative embodiments, aperture 35 and/or ferrule 36 may have other geometric shapes (e.g., rectangular, triangular, and hexagonal). However, annular shapes are particularly suitable for providing hermetic seals. Suitable materials for ferrule 36 include conductive materials such as annealed medical-grade titanium aluminum, stainless steel, and alloys thereof.

Feedthrough pin 38 is an electrically-conductive shaft that extends through ferrule 36 in an electrically-isolated arrangement. Suitable materials for feedthrough pin 38 include conductive materials such as niobium, which has a low resistivity, is compatible for welding with titanium, and has a low coefficient of expansion when heated. Suitable diameters for feedthrough pin 38 range from about 0.4 millimeters to about 0.6 millimeters. Such dimensions allow feedthrough pin 38 to be selected for low, medium, and high current applications.

During manufacture of battery 12, feedthrough assembly 24 is inserted within aperture 35 such that at least a portion of ferrule 36 extends outside of front housing 22a (i.e., outside of conductive cover 34). As a result, the volume taken up by ferrule 36 is located at least partially outside of battery housing 22, thereby increasing the amount of free space within battery housing 22. Ferrule 36 is secured to conductive cover 34 by welding (e.g., laser welding) or other suitable technique that provides an electrically-conductive contact between front housing 22a and ferrule 36.

Electrochemical cell 28 is placed within front housing 22a and cathode tabs 30 are coupled to feedthrough pin 38. This provides electrical contact between the cathode portion of electrochemical cell 28 and feedthrough pin 38. Rear housing 22b is then sealed to front housing 22a to form a hermetic seal laterally around battery 12. An electrolyte fluid is also introduced within battery 12 to promote ion transport within battery 12. Connector block 26 (not shown in FIG. 2) is then inserted onto ferrule 36 to provide a connection point for supplying power to an IMD.

FIG. 3 is a top rear perspective view of battery assembly 10, with a portion broken away to show the connection between cathode tabs 30 and feedthrough pin 38. Cathode tabs 30 and anode tab 32 extend through slots in insulative cup 33 of front housing 22a. When feedthrough assembly 24 is inserted into aperture 35, feedthrough pin 38 may be connected to cathode tabs 30 via coupling 40. Coupling 40 is a “U”-shaped element that is comprised of a conductive material, such as niobium. Coupling 40 may be secured to cathode tabs 30 and feedthrough pin 38 by welding or other similar technique. This provides the electrical connection between the cathode portion of electrochemical cell 28 and feedthrough pin 38.

Anode tab 32 may correspondingly be secured to conductive cover 34 (e.g., by welding) to provide an electrical connection between the anode portion of electrochemical cell 28 and conductive cover 34. Because ferrule 36 also electrical contacts front housing 22a, ferrule 36 is also electrically connected with the anode portion of electrochemical cell 28. However, because feedthrough pin 38 is electrically isolated from ferrule 36, an electrical short within battery 12 is prevented.

As shown in FIG. 3, a substantial portion of ferrule 36 is disposed outside of battery housing 22, increasing the amount of free space within battery housing 22. Accordingly, battery 12 may include additional active battery components to increase capacity. For example, electrochemical cell 28 may be increased in volume to provide additional storage capacity for battery 12. Alternatively, an additional cathode tab 30 may be incorporated to increase the electrical connection between the cathode portion of electrochemical cell 28 and feedthrough pin 38. In another alternative, an electrically-insulative collar (not shown) may be positioned around feedthrough pin 38 within battery housing 22. The electrically-insulative collar may reduce the risk of feedthrough pin 38 accidentally contacting conductive cover 34 and/or anode tab 32, and structurally supports feedthrough pin 38 within battery housing 22. Such alternative examples increase the durability of battery 12, without affecting capacity.

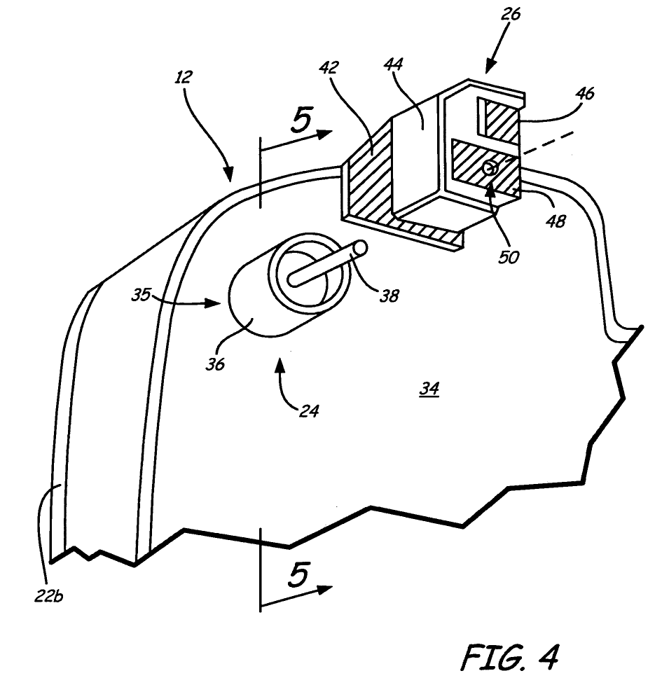

FIG. 4 is an expanded view of battery 12, illustrating the interaction between feedthrough assembly 24 and connector block 26. Connector block 26 includes base 42, main body 44, negative contact 46, positive contact 48, and orifice 50, where orifice 50 extends through main body 44 and positive contact 48. Main body 44 provides a housing for base 42, negative contact 46, and positive contact 48. Suitable materials for main body 42 include electrically-insulative materials, such as polyetherimides. Main body 44 also functions as an insulator to electrically isolate negative contact 46 from positive contact 48. Suitable materials for base 42, negative contact 46, and positive contact 48 include conductive materials, such as titanium, niobium, nickel, (e.g., gold-plated nickel), palladium, platinum, platinum-lawrencium alloys, iron-nickel-cobalt alloys commercially available under the trade designation “KOVAR” (from Carpenter Technology Corporation, Wyomissing, Pa.), and alloys thereof. While not visible in FIG. 4, electrical contact is made between base 42 and negative contact 46.

During manufacture of battery assembly 10, connector block 26 is aligned with feedthrough assembly 24. This illustrates another benefit of the present invention. Because ferrule 36 is at least partially disposed outside of battery housing 22, connector block 26 may be aligned with ferrule 36 for attaching connector block 26 to battery housing 22. If ferrule 36 were alternatively disposed within battery housing 22, connector block 26 would have to be aligned with aperture 35 prior to securing connector block 26 to battery housing 22. Such an alignment is tedious and time consuming, and increases the risk of misaligning connector block 26. In contrast, as shown in FIG. 4, connector block 26 may be readily fitted and retained over ferrule 36, which reduces time and skill required to manufacture battery assembly 10.

When connector block 26 is fitted over ferrule 36, feedthrough pin 38 extends through orifice 50, thereby creating an electrical connection between feedthrough pin 38 and positive contact 48. Similarly, ferrule 36 and conductive cover 34 electrically contact base 42, which correspondingly provides an electrical connection with negative contact 46. When connector block 26 is fully inserted over ferrule 36, feedthrough pin 38 may be welded (e.g., by laser welding) to positive contact 48, and base 42 may be welded to conductive cover 34.

Accordingly, after welding, negative contact 46 is electrically connected to the anode portion of electrochemical cell 28, and positive contact 48 is electrically connected to the cathode portion of electrochemical cell 28. As a result, connector block 26 provides a suitable location for connecting circuitry of an IMD (e.g., via ribbon bonding). In an alternative embodiment of the present invention, the connections between contacts 46 and 48, and the anode and cathode portions of electrochemical cell 28 may be reversed such that contact 46 is positive polarity and contact 48 is negative polarity.

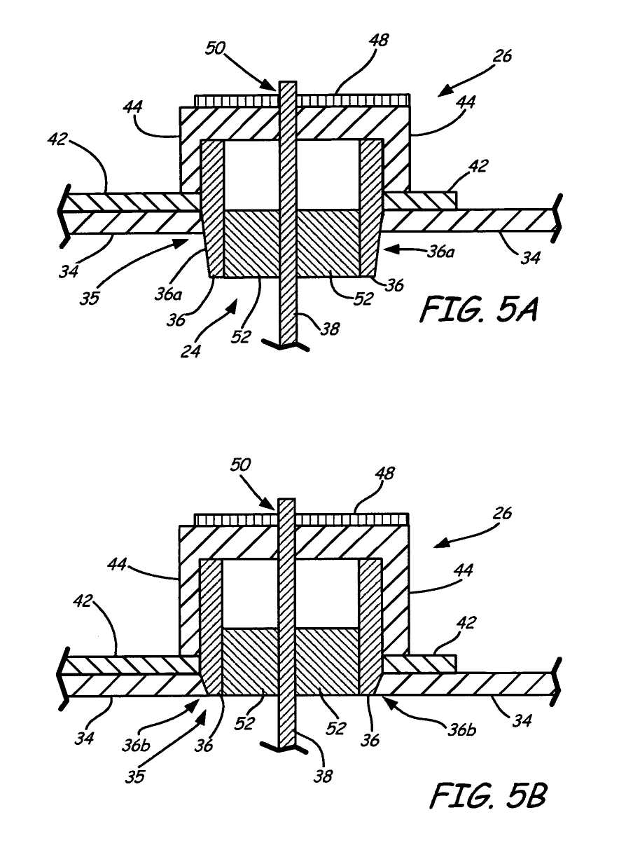

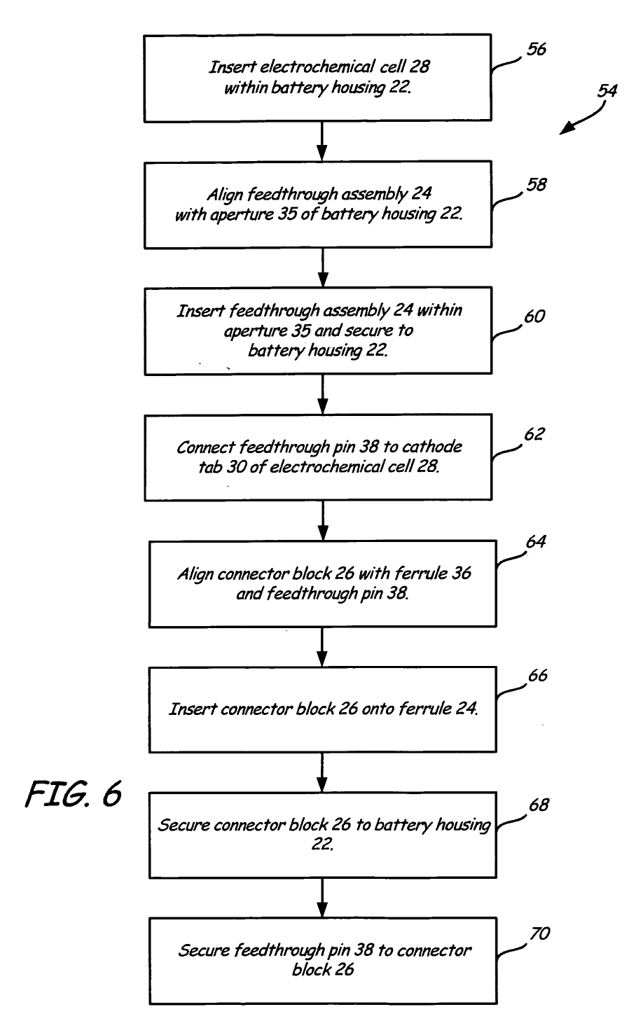

FIGS. 5A and 5B are sectional views of section 5-5 taken in FIG. 4, depicting alternative embodiments in which feedthrough assembly 24 and connector block 26 are secured to conductive cover 34. As shown in FIG. 5A, ferrule 36 extends within aperture 35, and is partially disposed outside battery housing 22. This embodiment is suitable for use with thin IMDs, where the height of connector block 26 is required to be low. While feedthrough assembly 24 remains partially within battery housing 22, an amount of free space corresponding to the volume of feedthrough assembly 24 extending outside of battery housing 22 is obtained. Additionally, as discussed above, feedthrough assembly 24 may be used to align connector block 26 to increase the manufacturing efficiency of battery assembly 10.

As further shown in FIG. 5A, ferrule 36 may include contoured portion 36a, which is a sloped surface that corresponds to a slope in aperture 35. Alternatively, contoured portion 36a may include a flanged edge that rests on conductive cover 34. Contoured portion 36a provides a frictional fit with aperture 35, and is beneficial for predetermining how far ferrule 36 extends outside of battery housing 22 when inserted through aperture 35. In general, the greater the slope of contoured portion 36a, the further ferrule 36 may extend outside of battery housing 22. In the embodiment shown in FIG. 5A, contoured portion 36a allows at least about 50% of the volume of ferrule 36 to be disposed outside of battery housing 22, and within connector block 26. Contoured portion 36a also allows ferrule 36 to be retained within aperture 35 during a welding process, which increases the ease of manufacturing.

Feedthrough assembly 24 also includes insulating seal 52, which electrically isolates feedthrough pin 38 from ferrule 36 and provides a hermetic seal within aperture 35. Suitable materials for insulating seal 52 include glass materials, such as CABAL-12 (calcium-boro-aluminate) glass. CABAL-12 is corrosion resistant as well as being a good insulator. Accordingly, CABAL-12provides for good insulation between feedthrough pin 38 and ferrule 36, as well as being resistant to the corrosive effects of the electrolyte fluid contained within battery 12.

During manufacture of battery assembly 10, ferrule 36, feedthrough pin 38, and the material for insulating seal 52 may be heated to melt the material for insulating seal 52, thereby forming hermetic seals within ferrule 34 and around feedthrough pin 38. While ferrule 36 is only partially filled with insulating seal 52, as shown in FIG. 5A, insulating seal 52 may alternatively fill the entire inner region of ferrule 36.

As shown in FIG. 5B, ferrule 36 extends within aperture 35, and is disposed almost completely outside of battery housing 22, such that the base of ferrule 36 is flush with the inner surface of conductive cover 34. Accordingly, the amount of free space obtained within battery housing 22 effectively corresponds to the volume of ferrule 36. Additionally, feedthrough assembly 24 may also be used to align connector block 26, thereby increasing the manufacturing efficiency of battery assembly 10.

In the embodiment shown in FIG. 5B, ferrule 36 includes contoured portion 36b, which is a sloped portion of ferrule 36, similar to contoured portion 36a, discussed above in FIG. 5A. Contoured portion 36b illustrates how a sharper angle allows ferrule 36 to extend further outside of battery housing 22.

In the embodiment shown in FIG. 5B, contoured portion 36a allows at least about 98% of the volume of ferrule 36 to be disposed outside of battery housing 22, and within connector block 26.

As generally illustrated in FIGS. 5A and 5B, ferrule 36 may be positioned at a variety of locations within aperture 35, which correspondingly allows a variety of different volumes of free space to be obtained within battery housing 22. However, because ferrule 36 extends within connector block 26, the overall volume of battery assembly 10 is preserved. Accordingly, battery assembly 10 is has increased volumetric efficiency for use with a variety of IMDs.

FIG. 6 is a block diagram illustrating battery manufacturing method 54, which includes steps 56-70. When manufacturing battery 12 pursuant to method 54, electrochemical cell 28 may initially be inserted within battery housing 22 (i.e., front housing 22a and rear housing 22b) (step 56). This electrically contacts anode tab 32 of electrochemical cell 28 with conductive cover 34.

Feedthrough assembly 24 may be manufactured prior to installation with battery housing 22. Feedthrough assembly 24 may be manufactured by inserting feedthrough pin 38 within ferrule 36, and placing an insulative material between ferrule 36 and feedthrough pin 38. The insulative material may then be melted and reformed to provide a hermetic seal between ferrule 36 and feedthrough pin 38, which also electrically isolates feedthrough pin 38 from ferrule 36.

Feedthrough assembly 24 is aligned with aperture 35 of front housing 22a (step 58). When properly aligned, feedthrough assembly 24 is inserted within aperture 35 such that at least a portion of ferrule 36 extends outside of battery housing 22 (step 60). Ferrule 36 of feedthrough assembly 24 is then secured to conductive cover 34 (e.g., via welding). This provides an electrical connection between the anode portion of electrochemical cell 28 and ferrule 36. Feedthrough pin 38 is then connected to cathode tabs 30 of electrochemical cell 28 (e.g., via welding) (step 62), which provides an electrically connection between the cathode portion of electrochemical cell 28 and feedthrough pin 38.

Connector block 26 is then aligned with the portion of ferrule 36 that extends outside of battery housing 22 (step 64). As discussed above, the external portion of ferrule 36 may be used as an alignment locator to properly identify where connector block 26 is to be installed. Connector block 26 may then be readily inserted onto ferrule 36 (step 66), and secured to battery housing 22 (step 68). Base 42 of connector block 26 is secured to battery housing 22 by welding or other suitable techniques for electrically connecting battery housing 22, ferrule 36, and base 42. Because base 42 electrically connects to negative contact 46, negative contact 46 is correspondingly electrically connected to the anode portion of electrochemical cell 28.

Feedthrough pin 38 is then secured to positive contact 48 of connector block 26 (e.g., via welding) to electrically connect positive contact 48 to the cathode portion of electrochemical cell 28 (step 70). Circuitry of an IMD may then be connected to negative contact 46 and positive contact 48 (e.g., via ribbon bonding) to receive power from battery 12. While steps 56-70 of method 54 are described in the order shown in FIG. 6, such steps are not intended to limited to such order, and may be performed in a variety of sequences. For example, feedthrough assembly 24 may be aligned with and inserted within aperture 35 (steps 58 and 60) before electrochemical assembly 28 is inserted within battery housing 22 (step 56). Additionally, front housing 22a and rear housing 22b may be secured together during method 54 as well. This involves sealing conductive cover 34 to the conductive outer casing of rear housing 22b, thereby hermetically sealing the interior portions of battery 12.

Although the present invention has been described with reference to preferred embodiments, workers skilled in the art will recognize that changes may be made in form and detail without departing from the spirit and scope of the invention.

1. A battery for use with implantable medical devices, the battery comprising:
a battery housing having an aperture;
a connector block connected to the battery housing over the aperture; and
a feedthrough assembly disposed through the aperture of the battery housing, the feedthrough assembly comprising:
a ferrule, wherein at least a portion of the ferrule extends outside of the battery housing; and
a feedthrough pin extending through the ferrule, the feedthrough pin being electrically connected to a first portion of the connector block.

2. The battery of claim 1, wherein the ferrule has a volume, and wherein at least about 50% of the volume extends outside the battery housing.

3. The battery of claim 2, wherein at least about 98% of the volume extends within the connector block.

4. The battery of claim 1, wherein the feedthrough assembly further comprises an electrically-insulative seal disposed within the ferrule to electrically isolate feedthrough pin from the ferrule.

5. The battery of claim 1, wherein the portion of the ferrule that extends outside of the battery housing is disposed within the connector block.

6. The battery of claim 1, wherein the ferrule is electrically connected to a second portion of the connector block.

7. The battery of claim 1, wherein the ferrule comprises a contoured portion.

8. The battery of claim 7, wherein the contoured portion determines the portion of the ferrule that extends outside of the battery housing.

9. A battery for use with implantable medical devices, the battery comprising:
a battery housing;
a connector block connected to the battery housing, the connector block having a first electrical contact and a second electrical contact; and
a feedthrough assembly comprising:
a ferrule disposed at least partially within the connector block, wherein the ferrule is electrically connected to the first contact of the connector block; and
a feedthrough pin extending through the ferrule, the feedthrough pin being electrically connected to the second contact of the connector block.

10. The battery of claim 9, further comprising an electrochemical cell disposed within the battery housing, the electrochemical cell having a first electrode and a second electrode, wherein the ferrule electrically connects the first electrode to the first contact of the connector block, and the feedthrough pin electrically connects the second electrode to the second contact of the connector block.

11. The battery of claim 9, wherein the ferrule defines a volume, and wherein at least about 50% of the volume extends within the connector block.

12. The battery of claim 11, wherein at least about 98% of the volume extends within the connector block.

13. The battery of claim 9, wherein the feedthrough assembly further comprises an electrically-insulative seal disposed within the ferrule to electrically isolate feedthrough pin from the ferrule.

14. The battery of claim 9, wherein the ferrule comprises a contoured portion.

15. A method of making a battery having a battery housing, the method comprising:
aligning a feedthrough assembly with an aperture of the battery housing, wherein the feedthrough assembly comprises a ferrule and a feedthrough pin electrically isolated from the ferrule;
inserting the feedthrough assembly within the aperture such that the ferrule extends at least partially outside the battery housing; and
inserting a connector block onto the ferrule of the feedthrough assembly, wherein the ferrule electrically contacts a first portion of the connector block and the feedthrough pin electrically connects a second portion of the connector block.

16. The method of claim 15, further comprising welding the connector block to the battery housing.

17. The method of claim 15, wherein a portion of the ferrule is disposed within the inserted connector block.

18. The method of claim 15, further comprises electrically connecting the feedthrough pin to a first electrode of an electrochemical cell that is disposed within the battery housing.

19. The method of claim 15, wherein the ferrule has a volume, and wherein at least about 50% of the volume extends outside the battery housing.

20. The method of claim 15, further comprising aligning the connector block with the ferrule.
